(19) United States
(12) Patent Application Publication
(10) Pub. No.: US 2009/0162325 A1
(43) Pub. Date: Jun. 25, 2009

(54) CELL IMPLANTATION TO PREVENT AND/OR TREAT AUTOIMMUNE DISEASE

(76) Inventors: Robert Bartlett Elliott, Auckland (NZ); Stephen John Martin Skinner, Auckland (NZ); Paul Lip Jin Tan, Auckland (NZ)

Correspondence Address: GREENLEE WINNER AND SULLIVAN P C, 4875 PEARL EAST CIRCLE, SUITE 200, BOULDER, CO, 80301, US

(21) Appl. No.: 11/916,963

(22) PCT Filed: Jun. 7, 2006
(86) PCT No.: PCT/NZ06/00141

§ 371 Date: May 28, 2008

(30) Foreign Application Priority Data
Jun. 8, 2005 (NZ) 540597

(51) Int. Cl. A61K 35/30; A61P 3/10; A61P 37/00

(52) U.S. Cl. 424/93.7

(57) ABSTRACT

The present invention is directed to the prevention or treatment of autoimmune diseases, and in particular, of type I diabetes, by administering a therapeutically effective amount of an implantable composition comprising living choroid plexus cells.

### FIELD OF THE INVENTION

The present invention is directed to the prevention and/or treatment of autoimmune disease, particularly although by no means exclusively, to the prevention and/or treatment of type I diabetes.

### BACKGROUND

Type I diabetes, also known as insulin-dependent diabetes mellitus (IDDM) or juvenile-onset diabetes, is an autoimmune disease whereby the body destroys its own insulin producing islet beta cells. By the time disease becomes evident about 80% of beta cells have been damaged or destroyed. The damage occurs due to chronic inflammation. The inflammation of the islets (insulitis) is due to a lymphocytic infiltrate of predominantly CD8 T cells, variable numbers of CD4 T cells, B cells, macrophages and natural killer cells. The expression of HLA Class I molecules on the islet cells are increased. The mechanisms of destruction of beta islet cells include a role for CD8 T cells, cytokines produced by cells of the inflammatory infiltrate such as interleukin 1, interleukin-6 and interferon alpha, and superoxide radicals and nitric oxide. (Atkinson M A and Eisenbarth GS. Type 1 Diabetes: new perspectives on disease pathogenesis and treatment Lancet 2001; 358: 221-229).

Destruction of the beta cells results in insufficient insulin being produced by the remaining islet cell population and a build up of glucose in the blood and urine. Such elevated blood glucose levels are responsible for many health problems associated with diabetes.

Diabetes affects over 18 million people in the United States alone, and of these, approximately 5 to 10% have type I diabetes. Currently there is no cure for type I diabetes and treatment usually requires the injection of insulin along with diet modifications to control blood glucose levels. Such treatment regimens can be difficult to manage and severely impact on a patient's lifestyle.

Alternative treatments include pancreatic transplantation. However, this involves complex surgical procedures and does not have a high success rate. More recent islet cell transplantation procedures appear to show promising results. However, such islet cell transplants require two or more donor pancreases to supply sufficient islet cells which places a major limitation on this therapy. Alternative sources of insulin secreting cells, include islets from pig pancreases, genetically modified liver (or other) cells that are able to secrete insulin, or stem cells that are cultured under conditions that favour differentiation into insulin secreting islet cells. Transplants using such alternative sources require additional clinical and/or ethical approval.

With autoimmune diseases, such as type I diabetes, cells are destroyed so that any treatment has to continue for the lifetime of the patient.

There are currently no prevention therapies available for people at risk of type I diabetes. Autoimmune type 1 diabetes is associated with T cell and antibody responses to autoantigens which include insulin, islet cell cytoplasmic antigens such as pancreatic sialoglycoconjugate tyrosine phosphatases IA-2 and IA-2β and glutamate decarboxylase. Autoantibodies are present for a moderate to long symptomless period (pre-diabetic phase) prior to clinical expression of disease. This suggests that it might be possible to develop an intervention to prevent disease. (American Diabetes association. Diagnosis and Classification of Diabetes Mellitus. Diabetes Care 2005; 28: S 37-S 42).

Some potential prevention therapies are in development, for example, genetically modified monoclonal antibodies (mAbs) are being designed that target factors that may be involved in the disease process, such as CD3. However, mAb therapies in general have been disappointing. Another potential therapeutic is insulin-like growth factor I (IGF-I) which regulates islet cells and protects against type I diabetes. However, this substance is unstable and a more suitable synthetic substance is in development.

It is therefore desirable to provide a method for preventing the onset of disease in patients at risk of developing autoimmune diseases, such as type I diabetes. It would also be desirable if such a method could also be used to treat patients with such diseases.

It is an object of the invention to go some way towards achieving these desiderata and/or to provide the public with a useful choice.

### SUMMARY OF THE INVENTION

The present invention provides a method for preventing the onset of type I diabetes in a patient at risk thereof, said method comprising administering to said patient a therapeutically effective amount of implantable composition comprising living choroid plexus cells.

The present invention further provides a method for delaying the onset of type I diabetes in a patient at risk thereof, said method comprising administering to said patient a therapeutically effective amount of an implantable composition comprising living choroid plexus cells.

The present invention further provides a method for treating type I or type II diabetes in a patient in need thereof, said method comprising administering to said patient a therapeutically effective amount of an implantable composition comprising living choroid plexus cells.

The present invention further provides a use of living choroid plexus cells in the manufacture of an implantable composition to prevent or delay the onset of type I diabetes in a patient in need thereof.

The present invention further provides a use of living choroid plexus cells in the manufacture of an implantable composition to treat type I or type II diabetes in a patient in need thereof.

The choroid plexus cell implants may be used in the present invention in combination with traditional treatment therapies for type I or type II diabetes. For example, in combination with insulin administration.

The choroid plexus cells may be combined with feeder or support cells to increase the viability of the implantable composition.

It is also contemplated that choroid plexus cells can be used to prevent or delay the onset of other autoimmune diseases and/or to treat such other autoimmune diseases.

It is also contemplated that neuronal cells other than choroid plexus cells, which have a neuronal factor secretory profile similar to choroid plexus cells, may be useful in the methods of the present invention.

The invention will be described in more detail by reference to the following figures.

### BRIEF DESCRIPTION OF THE FIGURES

FIGS. 1 and 2 show that the NT Cell treatment prevents Type I diabetes in the LtJ/NOD mouse model.

### DETAILED DESCRIPTION

The choroid plexus are lobulated structures comprising a single continuous layer of cells derived from the ependymal layer of the cerebral ventricles. One function of the choroid plexus is the secretion of cerebrospinal fluid (CSF). Cerebrospinal fluid fills the four ventricles of the brain and circulates around the spinal cord and over the convexity of the brain. The CSF is continuous with the brain interstitial (extracellular) fluid, and solutes, including macromolecules, are exchanged freely between CSF and interstitial fluid. In addition to the production of CSF, the choroid plexus has been associated with the formation of the CSF-blood barrier (Aleshire SL et al., “Choroid plexus as a barrier to immunoglobulin delivery into cerebrospinal fluid.” J. Neurosurg. 63:593-7, 1985). However, its broader function is the establishment and maintenance of baseline levels of the extracellular milleu throughout the brain and spinal cord, in part by secreting a wide range of growth factors into the CSF. Studies have reported the presence of numerous potent trophic factors within choroid plexus including TGFb, GDF-15, GDNF, IGF2, NGF, NT-3, NT-4, BDNF, VEGF, and FGF2 (for review see Johanson C E et al., “Choroid plexus recovery after transient forebrain ischemia: role of growth factors and other repair mechanisms.” Cell Mol Neurobiol. 20:197-216, 2000).

It has surprisingly been found that living choroid plexus cells are useful in preventing or delaying the onset of type I diabetes.

The present invention is therefore directed to a method of preventing or delaying the onset of type I diabetes by administering a therapeutically effective amount of an implantable composition comprising living choroid plexus cells to a patient in need thereof.

The choroid plexus cells may be from the same species as the host recipient patient, ie. allograft, or may be from a different species, ie. xenograft.

The preferred source of choroid plexus cells for clinical use is from bovine or porcine. Most preferably the source of the choroid plexus cells is from porcine and in particular, from the Auckland Island herd of pigs. These pigs are substantially microorganism free, and in particular have a very low PERV copy number, making them highly suitable as donors for xenotransplantation.

The choroid plexus cell may be obtained from embryonic (fetal), newborn (neonatal) and adult pigs.

Preferably, the choroid plexus cells are isolated from pigs aged from −20 to +20 days old.

Neonatal choroid plexus cells will be generally be preferred for xenotransplantation as their isolation is typically less problematic than their fetal counterparts, whilst their survival following isolation, for example, in tissue culture or following xenotransplantation, is commonly better than adult choroid plexus cells. For pigs, the neonatal period is generally held to be the first 7 to 21 days following birth.

Typically, embryonic porcine cells are isolated during selected stages of gestational development. For example, cells can be isolated from an embryonic pig at a stage of embryonic development when the cells can be recognized, or when the degree of growth and/or differentiation of the cells is suitable for the desired application. For example, the cells are isolated between about day twenty to about day twenty-five of gestation and birth of the pig.

The isolated choroid plexus cells for use in the invention can be maintained as a functionally viable cell culture. Examples of the methods by which choroid plexus cells can be cultured are presented in WO 01/52871; WO 02/32437; WO 2004/113516; WO 03/027270; WO 00/66188 and/or NZ 532057/532059/535131, incorporated herein in their entirety. Media which can be used to support the growth of porcine cells include mammalian cell culture media, for example, Dulbecco's minimal essential medium, and minimal essential medium. The medium can be serum-free but is preferably supplemented with animal serum such as fetal calf serum, or more preferably, porcine serum (ie autologous serum).

The isolated choroid plexus cells may be co-cultured with feeder or support cells, such as fibroblasts, Sertoli cells, splenocytes, macrophages, thymocytes etc. Such support or feeder cells secrete growth factors which enhance the viability of the choroid plexus cells.

The implantable compositions used in the present invention may comprise a combination of choroid plexus cells and one or more types of feeder or support cells. It is envisaged that such a composition will remain viable in vivo for sustained periods of time.

When isolated from a donor pig, the choroid plexus cells used in the invention retain their phenotype and/or are capable of performing their function. Preferably, isolated choroid plexus cells are capable of maintaining differentiated functions in vitro and in vivo, and adhering to substrates, such as culture dishes.

The feeder or support cells may be isolated from the same donor pig as the choroid plexus cells.

The implantable composition may comprise “naked” living choroid plexus cells together with a pharmaceutically acceptable carrier or excipient, or the choroid plexus cells may be encapsulated in a biocompatible hydrogel such as alginate. Isolation and encapsulation of choroid plexus cells in alginate is described in WO 00/66188 which is incorporated herein by reference. Preferably, the living choroid plexus cells are encapsulated in alginate. Such encapsulation acts to protect the choroid plexus cells from destruction by the recipient host's immune system.

The implantable composition may further comprise “naked” living feeder or support cells or the feeder or support cells may be encapsulated separately or together with the choroid plexus cells.

Preferably the implantable composition for use in the methods of the present invention comprises alginate capsules of approximately 500 to 700 microns in diameter and containing approximately 500 to 3,000 living choroid plexus cells per capsule. When feeder or support cells are present, the capsules will contain approximately 500-3,000 living feeder or support cells or will contain 500-3,000 feeder or support cells in combination with choroid plexus cells. The number of capsules that are implanted into a patient to give a therapeutic effect can vary depending on the age and weight of the patient as well as the interior dimensions of the site of implantation in the body. Typically, if the composition is to be implanted into the peritoneal cavity between 1,000 and 100,000 capsules may be implanted per kg body weight.

In any event, a physician, or skilled person, will be able to determine the actual dose which will be most suitable for an individual patient which is likely to vary with age, weight, sex and response of the particular patient to be treated. The above mentioned doses are exemplary of the average case and can, of course, be varied in individual cases.

Transplantation of the choroid plexus cells, and optionally support or feeder cells, can be achieved by way of routine techniques, for example, by suspending “naked” choroid plexus cells, and optionally support or feeder cells, in a suitable buffer followed by injection or infusion into a suitable body site. Preferably, encapsulated cells are injected into the peritoneal cavity of a patient.

In addition, the “naked” or encapsulated choroid plexus cells, and optionally support or feeder cells, may be introduced into an implantable device before transplantation into a patient. Such a device may comprise a subcutaneous implant device that allows development of a prevascularised allogenic collagen reservoir for the placement of the porcine choroid plexus cells and optionally support or feeder cells. Preferably, the implant device is cell-impermeable but protein or secreted factor-permeable, such as the “TheraCyte” device available from TheraCyte, Inc., Irvine, Calif. Alternatively, the porcine choroid plexus cells, and optionally support or feeder cells, may be incorporated or embedded in a support matrix which is host recipient compatible and which degrades into products which are not harmful to the host recipient. Natural or synthetic biodegradable matrices are examples of such matrices. Natural biodegradable matrices include collagen matrices. Synthetic biodegradable matrices include synthetic polymers such as polyanhydrides, polyorthoesters, and polylactic acid. These matrices provide support and protection for the cells in vivo.

It is envisaged that once implanted, compositions used in the methods of the present invention will be effective for between a few weeks to several months and possibly up to two years. The efficacy of the implanted composition can be monitored over time by monitoring one or more factors that are known to be secreted by the choroid plexus cells or by monitoring the maintenance of blood glucose levels, and thus the maintenance of a non-diabetic status in the patient. Should the efficacy of the implantable composition decline, it may be retrieved and replaced by a freshly prepared composition. Such retrieval and replacement of the therapeutic implantable composition may be carried out as often as necessary as part of the treatment regimen to maintain the therapeutic effect.

Choroid plexus cells are known to secrete numerous neurological secretory factors such as insulin-like growth factor, transforming growth factor alpha, retinoic acid and nerve growth factor. It is not known exactly which of the factors that are secreted by the choroid plexus cells are responsible for the therapeutic effect seen in the present invention. Nor is it known whether the one or more secretory factors act directly or indirectly via an endogenous cascade system (for example), ie the mechanism for action is unknown.

The main patient group that it is envisaged that will benefit from the present invention are those patients at risk of developing type I diabetes. For example, children with a family history of type I diabetes and those who test positive for three autoantibodies (islet cell antibody, glutamic acid decarboxylose antibody GADA, and insulin autoantibodies IAA) have a very high risk of developing type I diabetes (Zieler et al, Diabetes 1999; 48: 460-468).

In addition, patients who have recently developed the disease and are in the so called “honeymoon period” may benefit significantly from the present invention. The “honeymoon period”, when the need for exogenous insulin suddenly decreases, occurs when some of the patients remaining islet cells become active again. The islet cells originally stopped working because of the high blood sugar levels when diabetes was first diagnosed. With normal blood sugar levels during insulin treatment, the inactive islets regain their ability to make insulin. Unfortunately, the “honeymoon period” does not last long and within a few months to a year, the remaining islet cells are destroyed by the body's immune system and the patient requires permanent insulin injections (Novodisk website 2005).

The present invention is directed to the prevention or treatment of type I diabetes, via stabilization and preservation of the pancreatic islet cells. In patients, such as those who have already been diagnosed and prior to, or during the “honeymoon period”, the present invention aims to deter further islet cell destruction.

It is also contemplated that the present invention will be useful in the treatment of both type I and type II diabetes due to the ability to maintain the integrity and functionality of the body's islet cell population.

It is also contemplated that the present invention will be useful in combination with traditional diabetes treatment regimen, such as insulin administration. However, it is expected that a significant reduction in the frequency of administrations and/or in the dose of insulin required would be required in patients who received the choroid plexus implantable compositions of the invention.

It is also contemplated that choroid plexus cells can be used to prevent or delay the onset of autoimmune diseases other than type I diabetes, and/or to treat autoimmune diseases other than type I diabetes. For example Stiff Man syndrome (SMS) is a rare, autoimmune neurological disease which affects approximately 1 in 200,000 individuals—both males and females. This condition is characterised by progressive stiffness and painful spasms in the back and lower limbs. The condition appears to be linked to type I diabetes, for example some individuals with Stiff Man syndrome show an immune response to an enzyme called glutamic acid decarboxylase (GAD). Individuals with classic type I diabetes show a similar immune response. GAD is an important enzyme in the formation of a chemical messenger in the brain and spinal cord and also in the transmission of insulin. When a patient is developing SMS or type I diabetes, antibodies to GAD are produced which leads to its destruction, thus interrupting transmission.

Accordingly, the invention provides an implant composition comprising isolated porcine choroid plexus cells which are suitable for administration to a xenogeneic recipient. The implantable composition can be used to delay or prevent the onset of type I diabetes and/or other autoimmune diseases such as SMS; and/or to treat type I and type II diabetes and other autoimmune diseases such as SMS. The implantable composition used in the present invention may further comprise isolated feeder or support cells such as Sertoli cells or fibroblasts.

As used herein, the term “isolated” refers to cells which have been separated from their natural environment. This term includes gross physical separation from the natural environment, e.g., removal from the donor animal, and alteration of the cells' relationship with the neighboring cells with which they are in direct contact by, for example, dissociation.

As used herein, the term “porcine” is used interchangeably with the term “pig” and refers to mammals in the family Suidae. Such mammals include wholly or partially inbred pigs, preferably those members of the Auckland Island pig herd which are described in more detail in applicants co-pending New Zealand specification no. 539491, incorporated herein by reference.

The term “treating” as used herein includes reducing or alleviating at least one adverse effect or symptom of type I or type II diabetes. Examples of adverse effects or symptoms include high blood glucose, obesity, aberrant glucose sensitivity and/or glucose insensitivity, aberrant insulin levels, diabetic microvascular and macrovascular disease, aberrant lipase secretion, aberrant secretin levels, aberrant cholecystokinin levels, steatorrhea, aberrant gastrin levels, and aberrant cholinergic and/or adrenergic function.

Accordingly, the choroid plexus cells, and optionally support or feeder cells, are transplanted into a patient suffering from or predisposed to type I diabetes, or type II diabetes, in an amount such that there is at least a partial reduction or alleviation of at least one adverse effect or symptom of the disease, disorder or condition.

As used herein the terms “administering”, “introducing”, and “transplanting” are used interchangeably and refer to the placement of the choroid plexus cells into a subject, e.g., a xenogeneic subject, by a method or route which results in localization of the choroid plexus cells at a desired site. The choroid plexus cells can be administered to a subject by any appropriate route which results in delivery of the cells to a desired location in the subject where at least a portion of the cells remain viable. It is preferred that at least about 5%, preferably at least about 10%, more preferably at least about 20%, yet more preferably at least about 30%, still more preferably at least about 40%, and most preferably at least about 50% or more of the cells remain viable after administration into a subject. The period of viability of the cells after administration to a subject can be as short as a few days, to as long as a few weeks to months. Methods of administering, introducing and transplanting cells or compositions for use in the invention are well-known in the art. Cells can be administered in a pharmaceutically acceptable carrier or diluent.

The term “host” or “recipient” as used herein refers to mammals, particularly humans, suffering from or predisposed to type I diabetes or type II diabetes into which choroid plexus cells of another species are introduced or are to be introduced.

The term ‘comprising’ as used in this specification and claims means ‘consisting at least in part of’, that is to say when interpreting independent claims including that term, the features prefaced by that term in each claim all need to be present but other features can also be present.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The invention consists in the foregoing and also envisages constructions of which the following gives examples only.

### EXAMPLE 1

### Effect of Encapsulated Choroids Plexus Implants in NOD Mice

The NOD mice are a strain of mice that are predisposed to insulin-dependent diabetes characterised by a lymphocytic infiltration of the islets of Langerhans of the pancreas (insulitis) resulting in destruction of insulin producing β cells and a marked decrease in pancreatic insulin production. The inflammatory lesion in the pancreas is associated with T-cell and antibody responses to several autoantigens. The NOD mouse is therefore a laboratory model of autoimmune diabetes, ie of type I diabetes. (Tisch R, Yang X D, Singer S M, Liblau R S, Fugger L, McDevitt H O. Immune response to glutamic acid decarboxylase correlates with insulitis in non-obese diabetic mice. Nature 1993; 366: 15-17). The insulitis appears soon after weaning at about 4 weeks of age. Insulitis persists without evidence of disease and this pre-diabetic phase extends for several weeks. Disease as indicated by high blood glucose levels and the presence of glucose in the urine appears at various times after 12 weeks of age when significant loss of insulin producing cells compromises insulin production to the extent that glucose metabolism is impaired.

### 1. Preparation of Encapsulated Choroid Plexus (CP) Cells

Preparation of CP Secretory Cell Implants
This example relates to the preparation of choroid plexus secretory cells suitable for encapsulation and implantation. All procedures are carried out in “GMP” licensed facilities, including strict infection barriers.
Neonatal pigs were anaesthetized with ketamine (500 mg/kg) and xylazine (0.15 mg/kg) and killed by exsanguination. The brain was immediately removed and dissected through the midline to reveal the fork of the choroid vessels. The choroid plexus was extracted and placed in Hanks Balanced Salt Solution (HBSS, 0-4° C.) supplemented with 2% human serum albumin. The tissue was chopped finely with scissors, allowed to settle and the supernatant removed. Collagenase (Liberase, Roche, 1.5 mg/ml, in 5 ml HBSS at 0-4° C.) was added and the chopped tissues mixed, allowed to sediment at unit gravity (1×g) and the supernatant was again removed. Collagenase (1.5 mg/ml, in 15 ml HBSS at 0-4° C.) was added and the preparation warmed to 37° C. and stirred for 15-20 minutes. The digested material was triturated gently with a 2 ml plastic Pasteur pipette and passed through a 200 μm stainless steel filter.
The resulting neonatal pig preparations were mixed with an equal volume of RPMI medium supplemented with 2-10% neonatal porcine serum (prepared at Diatranz/LCT). The preparations were centrifuged (500 rpm, 4° C. for 5 minutes), the supernatant removed and the pellet gently re-suspended in 30 ml RPMI supplemented with serum. This procedure produced a mixture of epithelioid leaflets or clusters of cells, about 50-200 microns in diameter, and blood cells. Blood cells were removed by allowing the mixture to sediment at unit gravity for 35 minutes at 0-4° C., removing the supernatant and re-suspending. The preparation was adjusted to approximately 3,000 clusters/ml in RPMI with 2-10% serum and placed in non-adherent Petri dishes. Half of the media was removed and replaced with fresh media (5 ml) after 24 hours and again after 48 hours. By this time, most clusters assumed a spherical, ovoid or branched appearance.
The cells were then encapsulated in alginate as follows

Encapsulation
A counted sample of choroid plexus clusters are washed twice in HBSS supplemented with 2% human serum albumin and once in normal saline. The majority of supernatant is removed from above the sedimented clusters and alginate (1.7%) added in the ratio 1 ml per 40,000 clusters. The clusters are carefully suspended in alginate and pumped through a precise aperture nozzle to produce droplets which are displaced from the nozzle by either controlled air flow or by an electrostatic potential generated between the cell suspension exiting the nozzle and the receiving solution.
The stirred receiving solution contains sufficient calcium chloride to cause gelation of the droplets of alginate and cell cluster mixture. After the suspension has passed through the nozzle and the droplets collected in the calcium chloride solution, the gelled droplets are coated sequentially with poly-L-ornithine (0.1% for 10 min), poly-L-ornithine (0.05% for 6 min) and alginate (0.17% for 6 min). The gelled droplets are then treated with sodium citrate (55 mM for 2 min) to remove sufficient calcium from the interior of the gelled capsules to liquidise the contents. The poly-L-ornithine provides sufficient bonding for the capsule wall to remain stable.
The characteristics of the capsules thus produced are reproducibly of 500-700 microns in diameter (98-100%), are spherical (less than 2% are elliptical or otherwise miss-shapen). There are few broken capsules (less than 1%). Empty capsules, containing no CP clusters are typically less than 15%. The majority of the cell clusters within the capsules are 100-300 microns along their longest axis. Small clusters (less than 100 microns) are typically 5-13% and large clusters (greater than 300 microns along their longest axis) represent approximately 1-4% of the total.
After encapsulation the cell clusters were more than 90% viable as determined by Acridine Orange/Propidium Iodide staining.
   2. Implantation into NOD Mice
Litters of NOD mice at weaning (21 days of age) were separated into two groups of approximately equal numbers. One group was implanted with 500-2000 capsules (500-700 microns in diameter) directly into the peritoneal cavity. The capsules contained viable clusters of porcine choroids plexus cells (approximately 500-3,000 cells per capsule). The other control group was implanted with an equivalent number and volume of capsules containing no cells. This was repeated with new litters of weaned NOD mice until there were approximately 20 in each group.
One week after implantation, the mice were given a diabetogenic diet (Flohe et al, Cytokene 21: 149-154). They were tested for high urinary glucose from day 80 of age onwards. Those mice with measurable urinary glucose were monitored weekly for high blood glucose. Mice were defined as diabetics when urinary glucose was high and weekly blood glucose recorded at 13 mM or greater for two consecutive weeks. The diabetic mice were maintained in good health by small doses of insulin.

### 3. Results

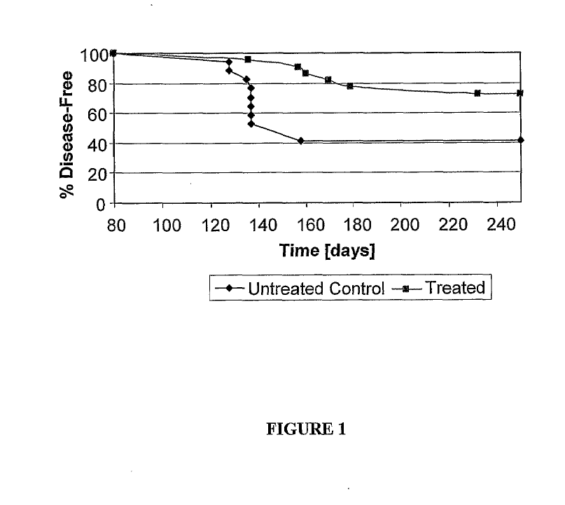

There was a clear decrease in the incidence of diabetes in the group that received encapsulated choroid plexus implantations compared to those that received empty capsules as shown in FIG. 1.
In this Experiment 9/17 of the control mice (implanted with empty capsules) became diabetic, with high urine and blood glucose (diamond shapes). In contrast, fewer of the treated mice (implanted with capsules containing living choroid plexus clusters from neonatal pigs), became diabetic ( 6/22) and, in those that became diabetic, the disease was significantly delayed.

### 4. Repeat Experiment

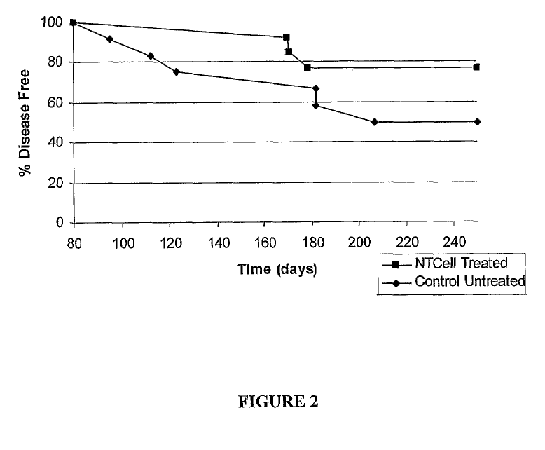

The experiment was repeated and in this repeat experiment 6/12 (50%) of the control mice (implanted with empty capsules) became diabetic, with high urine and blood glucose (FIG. 2, diamond shapes). In contrast, only 3/13 (23%) of the treated mice (implanted with capsules containing living choroid plexus clusters from neonatal pigs) became diabetic and, in those that became diabetic, the disease was significantly delayed.
Statistical analysis on the combined results from FIGS. 1 and 2 (n=36, treated; n=31, control) using the Chi-square test showed that treatment with choroid plexus cells significantly reduced the frequency of diabetes in NOD mice (P<0.0459).

### 5. Conclusion

The results of these studies clearly show that implanted choroid plexus cells are effective at protecting the health of pancreatic islets, and in particular, of protecting the insulin secreting beta cells and preventing the onset of diabetes.
Without wishing to be bound by theory, it is thought that the neurological factors that are secreted by the choroid plexus cells, such as neurotrophin NGF, insulin-like growth factor etc are involved in maintaining the islet cell structure. The islet cell is closely associated with a Schwann cell-like sheath and is innervated by direct connections with peripheral nerve cell bodies (Teitelman et al, J Neurobiol 34: 304-318 (1998); Tsui et al, Reviews in endocrine and metabolic disorders 4: 301-310 (2003)). It is thus contemplated that maintenance of the Schwann-like sheath and/or nervous connections of the islet insulin secreting beta cells, results in maintenance of the integrity and functionality of the islet beta cells per se.

### 5. Summary

The present invention shows for the first time that implantation of choroid plexus cells are effective at preventing the onset of diabetes in susceptible patient groups.

It is also contemplated that choroid plexus cell implantation will be equally effective at treating patients who have been diagnosed with early type I diabetes, or those patients who are experiencing the “honeymoon” period associated with type I diabetes.

It is also contemplated that choroid plexus cell implantation will be effective at treating patients with type II diabetes.

It is not the intention to limit the scope of the invention to the abovementioned examples only. As would be appreciated by a skilled person in the art, many variations are possible without departing from the scope of the invention.

For example, it is contemplated that neuronal cells other than choroid plexus cells that have a neuronal factor secretory profile similar to choroid plexus cells will also be useful in the methods of the present invention.

It is also contemplated that choroid plexus cell implantation will be useful in the prevention and treatment of autoimmune diseases, other than type I diabetes. This is particularly the case when the cells that are destroyed by the body's immune system are in close association with either a Schwann cell and/or are innervated by direct connections with nerve cell bodies.

It will be appreciated that it is not the intention to limit the scope of the invention to the abovementioned examples only. As would be appreciated by a skilled person in the art, many variations are possible without departing from the scope of the invention as set out in the accompanying claims.

### INDUSTRIAL APPLICATION

The present invention is useful in the prevention and treatment of diabetes which will have significant personal, social and economic benefits.

1-29. (canceled)

30. A method for preventing onset of type 1 diabetes in a patient at risk thereof, said method comprising administering to said patient a therapeutically effective amount of an implantable composition comprising living choroid plexus cells.

31. A method for delaying onset of type 1 diabetes in a patient at risk thereof, said method comprising administering to said patient a therapeutically effective amount of an implantable composition comprising living choroid plexus cells.

32. A method for treating type I or type II diabetes in a patient in need thereof, said method comprising administering to said patient a therapeutically effective amount of an implantable composition comprising living choroid plexus cells.

33. The method of claim 31, wherein said living choroid plexus cells are isolated from an adult, neonatal or fetal donor pig and the implantable composition comprises a xenograft.

34. The method of claim 33, wherein the living choroid plexus cells are isolated from a donor pig aged between −20 and +20 days.

35. The method of claim 31, wherein the implantable composition further comprises feeder or support cells.

36. The method of claim 35, wherein the feeder or support cells are selected from the group consisting of Sertoli cells, fibroblasts, splenocytes, macrophages and thymocytes.

37. The method of claim 35, wherein the feeder or support cells are isolated from the same donor pig as the choroid plexus cells.

38. The method of claim 31, wherein the implantable composition comprises “naked” choroid plexus cells and optionally “naked” feeder or support cells.

39. The method of claim 31, wherein the living implantable composition comprises encapsulated choroid plexus cells and optionally encapsulated feeder or support cells.

40. The method of claim 31, wherein the implantable composition is inserted in an implantable device prior to administration to said patient.

41. The method of claim 31, in combination with insulin administration.

42. The method of claim 31, in combination with insulin administration.

43. The method of claim 32, in combination with insulin administration.

44. A method for preventing or delaying the onset of an autoimmune disease, other than type I diabetes or any neurological autoimmune disease, comprising administering to said patient a therapeutically effective amount of an implantable composition comprising living choroid plexus cells.

45. A method for treating autoimmune diseases, other than type I diabetes or any neurological autoimmune disease, administering to said patient a therapeutically effective amount of an implantable composition comprising living choroid plexus cells.

46. The method of claim 44, wherein said autoimmune disease is Stiff Man syndrome.

47. The method of claim 44, wherein said composition is administered in an implant.

48. The method of claim 45, wherein said composition is administered in an implant.
